(19) United States
(12) Patent Application Publication
(10) Pub. No.: US 2024/0269639 A1
(43) Pub. Date: Aug. 15, 2024

(54) SUBSTRATE INCLUDING LOW TEMPERATURE CO-FIRED CERAMIC SUPPORT FOR DNA SYNTHESIS

(71) Applicant: Lenovo (Singapore) Pte. Ltd., Singapore (SG)

(72) Inventors: Marilia Santos Menossi Mortari, Butanta (BR); Bruno Marinaro Verona, Butanta (BR); Sergio Matias Pereira Junior, Butanta (BR); Caio Perez Gomes, Butanta (BR); Ariane Simoes Tunussi, Butantã (BR)

(21) Appl. No.: 18/162,361

(22) Filed: Jan. 31, 2023

(51) Int. Cl. B01J 19/00; B01J 31/12; B01J 31/14; B01J 35/04; B01J 37/02; C07H 1/00; C07H 21/04

(52) U.S. Cl. CPC B01J 19/0046; B01J 31/127; B01J 31/143; B01J 35/56; B01J 37/0217; B01J 37/0219; C07H 1/00; C07H 21/04; B01J 2219/00612; B01J 2219/00675; B01J 2219/00695; B01J 2219/00722

(57) ABSTRACT

Various aspects disclosed relate to a DNA synthesis substrate. the substrate includes a low temperature co-fired ceramic support. The substrate further includes a linker molecule functionalized to the low temperature co-fired ceramic support.

### BACKGROUND

Nucleic acid digital data storage is a robust approach to encoding and storing information over long periods of time, where data is stored at higher densities than magnetic tape or hard drive storage systems.

### SUMMARY OF THE INVENTION

Various aspects disclosed relate to a DNA synthesis substrate. The substrate includes a low temperature co-fired ceramic support. The substrate further includes a linker molecule functionalized to the low temperature co-fired ceramic support.

Various aspects disclosed relate to a DNA synthesis device. The device includes a reaction chamber. The device further includes a substrate located at least partially within the reaction chamber. The substrate includes a low temperature co-fired ceramic support. The substrate further includes a linker molecule functionalized to the low temperature co-fired ceramic support.

Various aspects disclosed relate to a method of synthesizing a single stranded DNA oligomer. The method includes contacting a first nucleotide a substrate. The substrate includes a low temperature co-fired ceramic support. The substrate further includes a linker molecule functionalized to the low temperature co-fired ceramic support. The method includes forming a bond between the linker and the first nucleotide. The method further includes contacting the first nucleotide with a second nucleotide. The method further includes forming a bond between the first nucleotide and the second nucleotide.

Various aspects disclosed relate to a method of making a substrate. The substrate includes a low temperature co-fired ceramic support. The substrate further includes a linker molecule functionalized to the low temperature co-fired ceramic support. The method includes hydroxylating a surface of the low temperature co-fired ceramic support. The method further includes contacting the hydroxylated surface of the low temperature co-fired ceramic support with the linker molecule. The method further includes bonding the linker molecule to a hydroxyl group of the hydroxylated surface of the low temperature co-fired ceramic support.

Various aspects disclosed relate to a DNA oligomer formed by contacting a first nucleotide a substrate. The substrate includes a low temperature co-fired ceramic support. The substrate further includes a linker molecule functionalized to the low temperature co-fired ceramic support. A bond between the linker and the first nucleotide. The first nucleotide is contacted with a second nucleotide. A bond is formed between the first nucleotide and the second nucleotide.

Various aspects disclosed relate to a device that synthesizes a single stranded DNA oligomer that encodes a set of information. The device includes a DNA synthesis substrate The substrate includes a low temperature co-fired ceramic support. The substrate further includes a linker molecule functionalized to the low temperature co-fired ceramic support. The device optionally further includes a reading device that interprets the single stranded DNA oligomer by decoding the interpreted single stranded DNA oligomer into the set of information. The reading device comprises a molecular electronics sensor that produces distinguishable signals in a measurable electrical parameter of the molecular electronics sensor, when interpreting the single stranded DNA oligomer.

### BRIEF DESCRIPTION OF THE FIGURES

The drawings illustrate generally, by way of example, but not by way of limitation, various aspects of the present invention.

FIG. 1 is a side-cross sectional view of microdevice or nanodevice used for DNA synthesis.

FIG. 2 is a scanned image of a surface of a substrate used for DNA synthesis.

FIG. 3 is an image showing DNA amplification of s ssDNA synthesized using the device.

### DETAILED DESCRIPTION OF THE INVENTION

Reference will now be made in detail to certain aspects of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than or equal to about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons or, in some aspects, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some aspects, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some aspects, the cycloalkyl group can have 3 to about 8-12 ring members, whereas in other aspects the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some aspects, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups. The term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain aspects there is no hydrocarbyl group. A hydrocarbylene group is a diradical hydrocarbon, e.g., a hydrocarbon that is bonded at two locations.

The term “DNA” refers to both biological DNA molecules and synthetic versions, such as made by nucleotide phosphoramidite chemistry, ligation chemistry or other synthetic organic methodologies. DNA, as used herein, also refers to molecules comprising chemical modifications to the bases, sugar, and/or backbone, such as known to those skilled in nucleic acid biochemistry. These include, but are not limited to, methylated bases, adenylated bases, other epigenetically marked bases, and non-standard or universal bases such as inosine or 3-nitropyrrole, or other nucleotide analogues, or ribobases, or abasic sites, or damaged sites. DNA also refers expansively to DNA analogues such as peptide nucleic acids (PNA), locked nucleic acids (LNA), and the like, including the biochemically similar RNA molecule and its synthetic and modified forms. All these biochemically closely related forms are implied by the use of the term DNA, in the context of the data storage molecule used in a DNA data storage system herein. Further, the term DNA herein includes single stranded forms, double helix or double-stranded forms, hybrid duplex forms, forms containing mismatched or non-standard base pairings, non-standard helical forms such as triplex forms, and molecules that are partially double stranded, such as a single-stranded DNA bound to a oligo primer, or a molecule with a hairpin secondary structure. Generally as used herein, the term DNA refers to a molecule comprising a single-stranded component that can act as the template for a polymerase enzyme to synthesize a complementary strand therefrom.

DNA sequences as written herein, such as GATTACA, refer to DNA in the 5′ to 3′ orientation, unless specified otherwise. For example, GATTACA as written herein represents the single stranded DNA molecule 5′-G-A-T-T-A-C-A-3′. In general, the convention used herein follows the standard convention for written DNA sequences used in the field of molecular biology.

The term “polymerase” refers to an enzyme that catalyzes the formation of a nucleotide chain by incorporating DNA or DNA analogues, or RNA or RNA analogues, against a template DNA or RNA strand. The term polymerase includes, but is not limited to, wild-type and mutant forms of DNA polymerases, such as Klenow, E. Coli Pol I, Bst, Taq, Phi29, and T7, wild-type and mutant forms of RNA polymerases, such as T7 and RNA Pol I, and wild-type and mutant reverse transcriptases that operate on an RNA template to produce DNA, such as AMV and MMLV.

The term “dNTP” refers to both the standard, naturally occurring nucleoside triphosphates used in biosynthesis of DNA (i.e., dATP, dCTP, dGTP, and dTTP), and natural or synthetic analogues or modified forms of these, including those that carry base modifications, sugar modifications, or phosphate group modifications, such as an alpha-thiol modification or gamma phosphate modifications, or the tetra-, penta-, hexa- or longer phosphate chain forms, or any of the aforementioned with additional groups conjugated to any of the phosphates, such as the beta, gamma or higher order phosphates in the chain. In general, as used herein, “dNTP” refers to any nucleoside triphosphate analogue or modified form that can be incorporated by a polymerase enzyme as it extends a primer, or that would enter the active pocket of such an enzyme and engage transiently as a trial candidate for incorporation.

The terms, “binary data” or “digital data” refers to data encoded using the standard binary code, or a base 2 {0,1} alphabet, data encoded using a hexadecimal base 16 alphabet, data encoded using the base 10 {0-9} alphabet, data encoded using ASCII characters, or data encoded using any other discrete alphabet of symbols or characters in a linear encoding fashion.

The term, “digital data encoded format” refers to a series of binary digits, or other symbolic digits or characters that come from the primary translation of DNA sequence features used to encode information in DNA, or the equivalent logical string of such classified DNA features. In some aspects, information to be archived as DNA may be translated into binary, or may exist initially as binary data, and then this data may be further encoded with error correction and assembly information, into the format that is directly translated into the code provided by the distinguishable DNA sequence features. This latter association is the primary encoding format of the information. Application of the assembly and error correction procedures is a further, secondary level of decoding, back towards recovering the source information.

The term, “distinguishable DNA sequence features” means those features of a data-encoding DNA molecule that, when processed by a sensor polymerase, produce distinct signals that can be used to encode information. Such features may be, for example, different bases, different modified bases or base analogues, different sequences or sequence motifs, or combinations of such to achieve features that produce distinguishable signals when processed by a sensor polymerase.

The term, a “DNA sequence motif” refers to both a specific letter sequence or a pattern representing any member of a specific set of such letter sequences. For example, the following are sequence motifs that are specific letter sequences: GATTACA, TAC, or C. In contrast, the following are sequence motifs that are patterns: G[A/T]A is a pattern representing the explicit set of sequences {GAA, GTA}, and G[2-5] is a pattern referring to the set of sequences {GG, GGG, GGGG, GGGGG}. The explicit set of sequences in the unambiguous description of the motif, while such pattern shorthand notations as those are common compact ways of describing such sets. Motif sequences such as these may be describing native DNA bases, or may be describing modified bases, in various contexts. In various contexts, the motif sequences may be describing the sequence of a template DNA molecule, and/or may be describing the sequence on the molecule that complements the template.

The term, “sequence motifs with distinguishable signals,” in the cases of patterns, means that there is a first motif pattern representing a first set of explicit sequences, and any of said sequences produces the first signal, and there is a second motif pattern representing a second set of explicit sequences, and any of said sequences produces the second signal, and the first signal is distinguishable from the second signal. For example, if motif G[A/T]A and motif G[3-5] produce distinguishable signals, it means that any of the set {GAA, GTA} produce a first signal, and any of the set {GGG,GGGG,GGGGG} produce a second signal, distinguishable from the first.

The term, “distinguishable signals” refers to one electrical signal from a sensor being discernably different than another electrical signal from the sensor, either quantitatively (e.g., peak amplitude, signal duration, and the like) or qualitatively, (e.g., peak shape, and the like), such that the difference can be leveraged for a particular use. In a non-limiting example, two current peaks versus time from an operating molecular sensor are distinguishable if there is more than about a 1×10⁻¹⁰ Amp difference in their amplitudes. This difference is sufficient to use the two peaks as two distinct binary bit readouts, e.g., a 0 and a 1. In some instances, a first peak may have a positive amplitude, e.g., from about 1×10⁻¹⁰ Amp to about 20×10⁻¹⁰ Amp amplitude, whereas a second peak may have a negative amplitude, e.g., from about 0 Amp to about −5×10⁻¹⁰ Amp amplitude, making these peaks discernably different and usable to encode different binary bits, i.e., 0 or 1.

The term, a “data-encoding DNA molecule,” or “DNA data encoding molecule,” refers to a molecule synthesized to encode data in DNA, or copies or other DNA derived from such molecules.

As used herein, electrodes refer to nano-scale conducting metal elements, with a nanoscale-sized gap between two electrodes in an individual pair of electrodes, and, in some aspects, comprising a gate electrode capacitively coupled to the gap region, which may be a buried or “back” gate, or a side gate. The electrodes may be referred to as “source” and “drain” electrodes in some contexts, or as “positive” and “negative” electrodes, such terminologies being common in electronics. Nano-scale electrodes will have a gap width between each electrode in a pair of electrodes in the 1 nm-100 nm range, and will have other critical dimensions, such as width and height and length, also in this range. Such nano-electrodes may comprise a variety of materials that provide conductivity and mechanical stability, such as metals, or semiconductors, for example, or of a combination of such materials. Examples of metals for electrodes include titanium and chromium. Although electrodes are refereed to as “nano-scale” it is within the scope of this disclosure for the electrode to have other dimensions. For example, electrodes can be micro-scale or macro-scale electrodes.

The term “silicate” as used herein refers to any silicon-containing compound wherein the silicon atom has four bonds to oxygen, wherein at least one of the oxygen atoms bound to the silicon atom is ionic, such as any salt of a silicic acid. The counterion to the oxygen ion can be any other suitable ion or ions. An oxygen atom can be substituted with other silicon atoms, allowing for a polymer structure. One or more oxygen atoms can be double-bonded to the silicon atom; therefore, a silicate molecule can include a silicon atom with 2, 3, or 4 oxygen atoms. Examples of silicates include aluminum silicate. Zeolites are one example of materials that can include aluminum silicate. A silicate can be in the form of a salt or ion.

The advent of digital computing in the 20^{th} Century created the need for archival storage of large amounts of digital or binary data. Archival storage is intended to house data for long periods of time, e.g., years, decades or longer, in a way that is very low cost, and that supports the rare need to re-access the data. Although an archival storage system may feature the ability to hold unlimited amounts of data at very low cost, such as through a physical storage medium able to remain dormant for long periods of time, the data writing and recovery in such a system can be the relatively slow or otherwise costly processes. The dominant forms of archival digital data storage that have been developed to date include magnetic tape, and, more recently, compact optical disc (CD). However, as data production grows, there is a need for even higher density, lower cost, and longer lasting archival digital data storage systems.

It has been observed that in biology, the genomic DNA of living organisms functions as a form of digital information archival storage. On the timescale of the existence of a species, which may extend for thousands to millions of years, the genomic DNA in effect stores the genetic biological information that defines the species. The complex enzymatic, biochemical processes embodied in the biology, reproduction and survival of the species provide the means of writing, reading and maintaining this information archive. This observation has motivated the idea that perhaps the fundamental information storage capacity of DNA could be harnessed as the basis for high density, long duration archival storage of more general forms of digital information.

What makes DNA attractive for information storage is the extremely high information density resulting from molecular scale storage of information. In theory for example, all human-produced digital information recorded to date, estimated to be approximately 1 ZB (ZettaByte) (˜10²¹ Bytes), could be recorded in less than 10²² DNA bases, or 1/60^{th} of a mole of DNA bases, which would have a mass of just 10 grams. In addition to high data density, DNA is also a very stable molecule, which can readily last for thousands of years without substantial damage, and which could potentially last far longer, for tens of thousands of years, or even millions of years, such as observed naturally with DNA frozen in permafrost or encased in amber.

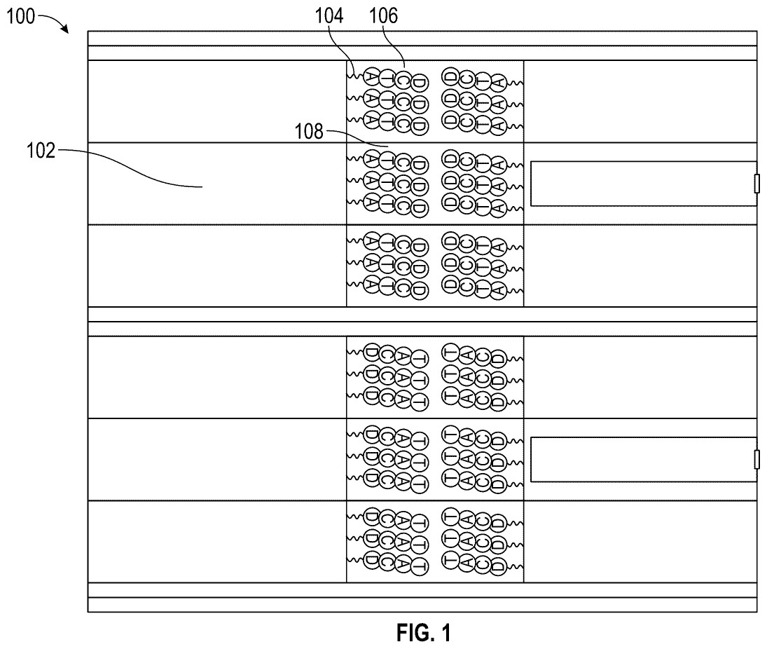

FIG. 1 is a side-cross sectional view of microdevice or nanodevice 100 used for DNA synthesis. Microdevice or nanodevice 100 includes support 102, linker molecule 104, nucleotide oligomer 106, and reaction chamber 108. As shown, support 102 forms the bounds reaction chamber 108. Additionally support 102 is the bulk material of device 100, but in some examples, support 102 can form only reaction chamber 108 while another material forms the bulk of device 100. Linker molecule 104 is functionalized or bonded to support 102. For example, a covalent bond may be formed between support 102 and linker molecule 104. Nucleotide oligomer 106 is bonded or functionalized to a linker molecule 104. Nucleotide oligomer 106 and linker molecule 104 can have a phosphodiester bond formed therebetween. Microdevice or nanodevice 100, can be any suitable DNA synthesis device. Examples of DNA synthesis devices include, without limitation, a microarray. Microdevice or nanodevice 100 can be an automated device.

Depending on the type of device that microdevice or nanodevice 100 is, reaction chamber 108 can be one of many different constructions. As shown in FIG. 1, reaction chamber 108 is a channel. In other constructions, reaction chamber 108 can be a well (e.g., if device 100 is a microarray), a cartridge, a pore, a reaction site. Microdevice or nanodevice 100 can include as few as one reaction chamber 108. In other examples, microdevice or nanodevice 100 can include any plural number of reaction chambers 108. For example, microdevice or nanodevice 100 can include 1 to 500 reaction chambers 108, 5 to 250 reaction chambers 108, 40 to 100 reaction chambers 108, less than, equal to, or greater than 2 reaction chambers 108, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500. Where multiple reaction chambers 108 are present, each reaction chamber 108 can have substantially the same dimensions. Alternatively, at least two reaction chambers 108 may differ in their dimensions. For example, one reaction chamber 108 may be shaped as a well, whereas another reaction chamber 108 may be formed as a channel.

Support 102 is formed from a low temperature co-fired ceramic material. Support 102 can include 100 wt % low temperature co-fired ceramic material. Alternatively, support 102 can include about 50 wt % to about 100 wt % low temperature co-fired ceramic material. As generally understood, low temperature co-fired ceramic materials are a branch of ceramic packaging substrates that meet the technical requirements for multi-chip assembly or single-chip packaging of low frequency, digital, RF, and microwave devices with their excellent electrical, mechanical, thermal, and process characteristics.

Low temperature co-fired ceramic materials has the advantages of high frequency characteristics, thermal stability, passive component integration. For example, low temperature co-fired ceramic materials have excellent high frequency, high Q characteristics and high-speed transmission characteristics. Low temperature co-fired ceramic materials have good temperature characteristics, can adapt to the characteristics of high current and high temperature resistance requirements. Low temperature co-fired ceramic materials are easy to achieve multi-function and improve assembly density, high reliability, high temperature, high humidity, shock vibration, can be applied to harsh environments. Additionally, in the frequency range of 2.4 MHz-80 GHz, the signal loss caused by low temperature co-fired ceramic technology is much lower than that of multilayer line technology.

(5) Due to the introduction of mass production equipment and process, the module size is reduced by 20%˜40%, and the cost can be greatly reduced.

Therefore, low temperature co-fired ceramic technology is generally considered to be a promising technology for future integrated components and substrate materials for high-frequency applications. At present, there are three major types of low-temperature co-fired ceramic materials: microcrystalline glass systems, glass+ceramic composite systems, and amorphous glass systems.

Microcrystalline glass is a composite of a large number of tiny crystals and a small amount of residual glass phase made from a certain composition of glass by controlled crystallization. It has the characteristics of easy to adjust the formula, simple process, and better performance. Such as low dielectric loss, suitable for the production of working frequency in 20˜30 GHZ devices, cordierite (2MgO—2Al₂O₃—5SiO₂), calcium silica (CaO, SiO₂), and lithium pyroxene (Li₂O—Al₂O₃—4SiO₂) is most widely used. The microcrystalline glass according to the composition of the base glass can generally be divided into silicate system, aluminosilicate system, borosilicate system, borate system, phosphate system, and other five categories. Microcrystalline glass uses silicate type of glass-ceramic materials, adding one or more oxides, such as P₂O₅, Li₂O, B₂O₃, ZrO₂, ZnO, TiO₂, SnO₂, sintering temperature in 850˜1050° C., small dielectric constant and thermal expansion coefficient.

Glass+ceramic composite systems are commonly used as an low temperature co-fired ceramic material. Adding low melting point glass phase in the ceramic, the glass softens during sintering and the viscosity decreases, which can reduce the sintering temperature. Glass is mainly a variety of crystallized glass, ceramic filling phase is mainly Al₂O₃, SiO₂, cordierite, mullite ceramic, or the like. The sintering temperature is around 900° C., the process is simple and flexible, easy to control and adjust the sintering characteristics and physical properties of the composite material, the dielectric constant and its temperature coefficient are small, the resistivity is high and the chemical stability is good.

In an amorphous glass system, the oxides that form the glass are mixed thoroughly, calcined between 800-950° C., then ball-milled and sieved, and sintered into dense ceramic substrates according to the ceramic process molding. This system is a simple process, the composition is easy to control, but the comprehensive performance of the ceramic substrate is less desirable, such as lower mechanical strength, dielectric loss is large, and is rarely used.

Non-limiting examples of suitable low temperature co-fired ceramic materials that can be used include a mixture of Al₂O₃, forsterite, and borosilicate glass; a mixture of borosilicate glass, SiO₂, and Al₂O₃; a mixture of BaO, SiO₂, and Al₂O₃; CaZrO₃; a mixture of BaO, B₂O₃, Al₂O₃, CaO, and SiO₂; a mixture of Nd₂O₃, TiO₂, and SiO₂; a mixture of PbO, Al₂O₃, and SiO₂; a mixture of CaO, Al₂O₃, SiO₂, B₂O₃, and Al₂O₃; a mixture of Al₂O₃, CaO, SiO₂, ZrO₂, MgO, B₂O₃; or a mixture of Al₂O₃, SiO₂, ZrO₂, and MgO.

While there are certainly many types of low temperature co-fired ceramic materials, the preparation of the ceramic material is generally divided into two methods, namely, the high-temperature melting method and chemical preparation method. The high-temperature melting method is to mix various oxides in a predetermined proportion, the liquid phase reaction in a high-temperature melting furnace (generally higher than 1400° C.), after water quenching, and finally ball milling or ultrasonic crushing, to obtain glass-ceramic powder; chemical preparation method is to dissolve different proportions of oxides and reactants into a specific solution, after the reaction to produce precipitation, the precipitate for the glass-ceramic powder, is used to produce the powder.

The chemical process involves the preparation and formulation of a ceramic slurry, which is cast into raw ceramic strips of up to several millimeters. The raw ceramic strip is then cut into small individual pieces and the desired through-holes are punched by mechanical or laser methods. In the next step, metallic conductors (Cu, Ag, Au, etc.) are filled with holes in the raw porcelain tape using techniques such as screen printing and micro-hole grouting, and conductive patterns are created. Finally, the single layer of raw porcelain tape is stacked together according to the process requirements, combined together by uniaxial and isostatic pressure lamination, low temperature (900-1000° C.) sintering molding, and finally made into a high density integrated circuit, also can be built-in passive components, in its surface, mount IC and active components, made of passive/active hybrid integrated functional modules.

Due to the excellent performance of low temperature co-fired ceramic, it has been successfully used in the manufacture of integrated circuit packages, multi-chip modules (MCM), microelectromechanical systems (MEMS), various chip inductors, chip capacitors, chip transformers, and chip antennas. Application areas include communication, automotive electronics, medical electronics, aerospace, and military electronics.

Features and Advantages of low temperature co-fired ceramic. Indeed low temperature co-fired ceramic materials tend to have the following characteristics: high resistivity to ensure insulation between signal lines; low dielectric constant to improve the signal transmission rate; low dielectric loss; low sintering temperature, can be co-fired with Cu.Ag and other high conductivity metals; low sintering shrinkage to ensure the accuracy of the circuit design; duitable coefficient of thermal expansion to ensure compatibility with silicon and other chips; and high thermal conductivity to prevent overheating of the substrate.

Although the use of low temperature co-fired ceramic materials is established in electronic devices, their use in biotechnology applications is not developed. Specifically, there is no indication that an low temperature co-fired ceramic material could or should function as a support for a DNA synthesis device such as microdevice or nanodevice 100.

As shown in FIG. 1, low temperature co-fired ceramic support 102 forms the boundaries of reaction chamber 108. In some examples, however, low temperature co-fired ceramic support 102 may cover less than 100% of the total surface area of reaction chamber 108. For example, low temperature co-fired ceramic can cover about 10% to about 95% of the total surface area of reaction chamber 108, about 70% to about 90%, less than, equal to, or greater than about 10%, 15, 20, 25, 30, 35, 40, 45, 50, 55, 60, 65, 70, 75, 80, 85, 90, or about 95% of the total surface area of reaction chamber 108.

Low temperature co-fired ceramic support 102 can be formed as an integral piece of microdevice or nanodevice 100. It is also possible to “retrofit” low temperature co-fired ceramic support 102 to an existing DNA synthesis device. For example, a reaction chamber of an existing device can be modified to be coated with the low temperature co-fired ceramic material.

One property that makes low temperature co-fired ceramic support 102 desirable to use in microdevice or nanodevice 100, is that it can be functionalized to react with linker molecule 104. As used herein “functionalized” is meant to refer to a surface of low temperature co-fired ceramic support 102 being bonded with terminal hydroxyl groups. About 50% to about 100% of the total surface area of the low temperature co-fired ceramic support 102 is functionalized, about 75% to about 90% surface area, less than, equal to, or greater than about 50%, 55, 60, 65, 70, 75, 80, 85, 90, 95, or about 100% of the total surface area of the low temperature co-fired ceramic support 102 is functionalized.

Functionalizing the surface of low temperature co-fired ceramic support 102 allows for a bond to be formed between low temperature co-fired ceramic support 102 and linker molecule 104. Linker molecule 104 includes a silicon. As an example, linker molecule can include a functionalized compound according to Formula I:

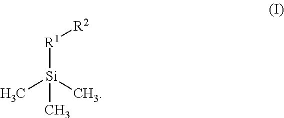

In Formula I, R¹ is selected from the group consisting of a bond or (C₁-C₂₀)hydrocarbyl. R² is selected from the group consisting of —OH and N(R³)₂. At each instance, R³ is independently selected from the group consisting of —H or substituted or unsubstituted (C₁-C₂₀)hydrocarbyl. In further examples, R¹ is selected from the group consisting of a bond or substituted or unsubstituted (C₂-C₇)hydrocarbyl. In still further examples, R¹ is selected from the group consisting of a bond or substituted or unsubstituted (C₂-C₂₀)alkenyl, substituted or unsubstituted (C₂-C₂₀)aryl, substituted or unsubstituted (C₂-C₂₀)hydroxyl, substituted or unsubstituted (C₂-C₂₀)alkyl, or substituted or unsubstituted (C₂-C₂₀)cycloalkyl.

In some examples, linker molecule 104 is a functionalized 3-aminopropyltriethoxysilane or a functionalized trimethylsilanol. Low temperature co-fired ceramic 102, can have the same linker molecules 104 attached thereto. It is also possible for low temperature co-fired ceramic 102 to have a mixture of different linker molecules 104.

Nucleotide oligomer 106 is functionalized (e.g., bonded) to linker molecule 104. Nucleotide oligomer 106 is a single-stranded DNA oligomer. Nucleotide oligomer 106 can range in length from about 2 bases to about 300 bases. The nucleotide oligomer can bases chosen from adenine, cytosine, guanine, and thymine. When in their reactant form (e.g., before polymerizing to form the oligomer), individual nucleotides are dNTPs.

Nucleotide oligomer 106 is synthesized in microsystem or nanosystem 100. Which is any device that takes a given set of DNA sequences and synthesizes DNA molecules from these sequences (see, for e.g.: Kosuri and Church, “Large Scale de novo DNA synthesis: technologies and applications. Nature Methods, 11: 499-509, 2014). Non-limiting examples of methods and devices for synthesizing nucleotide oligomer 106 include commercial technology offered by Agilent Technologies and Twist BioScience. For each desired sequence, multiple DNA molecules representing that sequence are produced. The multiplicity of molecules produced can be in the ranges of 10's, 100's, 1000's, millions or even billions of copies of DNA molecules for each desired sequence. All of these copies representing all the desired sequences may be pooled into one master pool of molecules. It is typical of such DNA writing systems that the writing is not perfect, and if N molecules are synthesized to represent a given input sequence, not all of these will actually realize the desired sequence. For example, they may contain erroneous deletions, insertions, or incorrect or physically damaged bases.

The sequence of nucleotide oligomer 106 can be determined by an encoder/decoder, which includes an algorithm with two functions: the encoder portion translates given digital data encoded format (e.g., digital/binary information) into a specific set of DNA sequences that are inputs to the DNA writer. The decoder portion translates a given set of DNA sequences of the type provided by the DNA reader, back into digital information.

In various aspects, a DNA reader may optionally be present system comprises substantially lower instrument capital costs, and higher per-base reading speed, and greater scalability in total number of reads per run, compared to currently available optical next generation sequencing instruments. In various aspects, the reading device for use herein is based on a CMOS chip sensor array device in order to increase the speed and scalability and decrease the capital costs. An aspect of such a device comprises a CMOS sensor array device, wherein each sensor pixel contains a molecular electronic sensor capable of reading a single molecule of DNA without any molecular amplification or copying, such as PCR, required. In various aspects, the CMOS chip comprises a scalable pixel array, with each pixel containing a molecular electronic sensor, and such a sensor comprising a bridge molecule and polymerase enzyme, configured so as to produce sequence-related modulations of the electrical current (or related electrical parameters such as voltage, conductance, etc.) as the enzyme processes the DNA template molecule.

Nucleotide oligomer 106 is read with a DNA reading device is a device that takes a pool of DNA molecules and produces a set of measured DNA sequences for molecules sampled or selected from this pool. Such readers actually survey only a small portion of the DNA molecules introduced into the system, so that only a small fraction will undergo an actual read attempt. It is further typical of such DNA reading devices that a given DNA molecule that is processed may not be read with entire accuracy, and thus there may be errors present in the read. As a result, it is also typical that the measured sequence outputs include various forms of confidence estimates and missing data indicators. For example, for each letter in a measure sequence, there may be a confidence probability or odds that it is correct, versus the other three DNA letter options, and there may be missing data indicators that indicate the identity of a letter is unknown, or there may be a set of optional sequence candidates with different probabilities representing a portion of a read.

The information encoder/decoder can be selected based on the properties of the DNA writer and DNA reader devices, so as to minimize or reduce some overall measure of the cost of the information storage/retrieval process. One key component of system cost is the overall error rate in retrieved information.

In general, a DNA writer device can introduce writing errors, and a DNA reading device can produce reading errors, and so the processes of storing information in the system and then later retrieving it potentially results in an error rate seen in the retrieved information. The encoder/decoder algorithm can be chosen to minimize or reduce this error rate, based on the error properties and propensities of the DNA reader and DNA writer.

In various aspects, nucleotides can be preferentially selected for incorporation in nucleotide sequences based on their ease of synthesis in the writing process that forms molecules, reduced propensity to form secondary structure in the synthesized molecules, and/or ease in reading during the data decoding process. In various aspects, bad writing motifs and bad reading motifs are avoided in the selection of nucleotides for incorporation into nucleotide sequences, with a focus on incorporating segments in the nucleotide sequence that will produce mutually distinguishable signals when that nucleotide sequence is read to decode the encoded information. For example, in reading a nucleotide sequence, A and T are mutually distinguishable, C and G are mutually distinguishable, A, C and G are mutually distinguishable, AAA and TT are mutually distinguishable, A, GG and ATA are mutually distinguishable, and C, G, AAA, TTTT, GTGTG are mutually distinguishable. These, and many other sets of nucleotide and nucleotide segments provide mutually distinguishable signals in a reader, and thus can be considered for incorporation in a nucleotide sequence when encoding a set of information into a nucleotide sequence.

Additionally, there are nucleotide segments that are difficult to write, and thus should be avoided when encoding a set of information into a nucleotide sequence. In various aspects, encoding of a set of information into a nucleotide sequence comprises the use of one of the remaining distinguishable feature sets as the encoding symbols, such as may correspond to binary 0/1, trinary 0/1/2 or quad 0/1/2/3 code, etc., along with an error correcting encoding to define the set of information in a way that avoids the hard to read and hard to write features. In this way, overall performance of an information storage system is improved.

In general, in order to reduce errors, the digital data encoding/decoding algorithm can comprise error detecting and error correcting codes selected to minimize error production, given the actual error modes of the DNA writer and DNA reader. These codes can be devised with the benefit of prior knowledge of the error modes, i.e., the propensity for particular errors of the writer and reader.

In various aspects, the error correcting codes reside within a single nucleotide sequence. For example, one segment of binary data is encoded in one DNA sequence, with the use of error correction and/or detection schemes on the DNA side. Such schemes may also involve encoding one segment of binary data into multiple DNA sequences, to provide another level of redundant encoding of information, which is analogous to error correction through redundant storage. Error detection schemes include, but are not limited to, repetition code, parity bits, checksums, cyclic redundancy checks, cryptographic hash functions, and error correcting codes such as hamming codes. Error correction schemes include, but are not limited to, automatic repeat request, error correcting code such as convolutional codes and block codes, hybrid automatic repeat request, and Reed-Solomon codes.

In various aspects, a method of devising an optimal or highly efficient error correcting encoding, wherein the incoming digital data is considered as binary words of length N, comprises the steps of: providing a space of all DNA words of length M, such that there are many more possible DNA words than binary words (i.e., 4^{M}>>2^{N}); and selecting a subset of 2^{N} of the DNA words to use as code words for encoding the 2^{N} binary information words, such that when each of these DNA code words is expanded into the set of probable DNA writing errors for the given word, and then that set further expanded by the set of probable reading errors words, these resulting 2^{N} sets of DNA words remain disjoint with high probability. In such a case, any word read by the reader can be properly associated back to the ideal encoded DNA word with very high probability. This method constitutes a combination of error correcting and error avoiding encoding of information. In addition, the decoding algorithm would also naturally make use of confidence or odds information supplied by the reader, to select the maximum likelihood/highest confidence decoding relative the encoding scheme.

Another key aspect of optimizing the overall DNA data storage system costs is the time required to write data. For example, the critical time cost in many aspects may be the time cost of writing the data. In various aspects, the writing of certain slow-to-synthesize bases and sequence motifs are avoided in order to shorten the overall writing time. In other aspects, the writing is faster, such as by reducing the time spent on each chemistry cycle of some cyclical process that writes one base in many parallel synthesis reactions, with acceptance of a higher overall writing error rate.

Similarly, for reading, a faster reading process may be employed, with the trade-off being a higher rate of reading errors. In various examples, a faster reading process is employed without an increase in error by avoiding the introduction of certain types of sequences in the encoding that are difficult to read at a rapid rate, such as homopolymer runs. In either case, the information encoding/decoding algorithm can be co-optimized with these choices that allow for faster reading/writing but with extra error modes to be avoided, or avoiding slow-to-read/write sequence motifs, handled within the encoding/decoding.

In various aspects of the DNA information storage system herein, the DNA reading device comprises a massively parallel DNA sequencing device, which is capable of a high speed of reading bases from each specific DNA molecule such that the overall rate of reading stored DNA information can be fast enough, and at high enough volume, for practical use in large scale archival information retrieval. The rate of reading bases sets a minimum time on data retrieval, related to the length of stored DNA molecules.

In various aspects, a molecular electronics sensor extracts information from single DNA molecules, in a way that provides a reader for digital data stored as DNA. A molecular electronics sensor comprises a circuit in which a single molecule, or a complex of a small number of molecules, forms a completed electrical circuit spanning the gap between a pair of nano-scale electrodes, and an electronic parameter is modulated by this single molecule or complex, and in which this parameter is measured as a signal to indicate (“sense”) the single molecule or complex interacting with target molecules in the environment. In various aspects, the measured parameter is current passing through the electrodes, versus time, and the molecular complex is conjugated in place with specific attachment points to the electrodes.

A polymerase used for synthesis of the nucleotide oligomer can be a native or mutant form of Klenow, Taq, Bst, Phi29 or T7, or may be a reverse transcriptase. In various aspects, the mutated polymerase forms will enable site specific conjugation of the polymerase to the bridge molecule, arm molecule or electrodes, through introduction of specific conjugation sites in the polymerase. Such conjugation sites engineered into the protein by recombinant methods or methods of synthetic biology may, in various aspects, comprise a cysteine, an aldehyde tag site (e.g. the peptide motif CxPxR), a tetracysteine motif (e.g., the peptide motif CCPGCC) (SEQ ID NO: 1), or an unnatural or non-standard amino acid (NSAA) site, such as through the use of an expanded genetic code to introduce a p-acetylphenylalanine, or an unnatural cross-linkable amino acid, such as through use of RNA- or DNA-protein cross-link using 5-bromouridine, (see, e.g., Gott, J. M., et al., Biochemistry, 30 (25), pp 6290-6295 (1991)).

A cloud based DNA data archival storage system can be used in conjunction with microscale or nanoscale system 100, in which the complete reader system in certain aspects, deployed in aggregate format to provide the cloud DNA reader server of the overall archival storage and retrieval system. A cloud computer system can include a standard storage format. Such as standard cloud computer system comprises a DNA archival data storage capability as indicated. In various aspects, a cloud-based DNA synthesis system can accept binary data from the cloud computer and produce the physical data encoding DNA molecules. This server stores the output molecules in a DNA data storage archive wherein the physical DNA molecules that encode data are stored in a dried or lyophilized form, or in solution, at ambient temperature, cooled temperature, or frozen. When data is to be retrieved, a sample of the DNA from the archive is provided to the DNA data reader server, which outputs decoded binary data back to the primary cloud computer system. This DNA data reader server is, in certain aspects, powered by a multiplicity of DNA reader chip-based systems in combination with additional computers that perform the final decoding of the DNA derived data back to the original data format of the primary cloud storage system.

Search of an archive for a literal input string can be achieved by encoding the search string or strings of interest into DNA form, synthesizing a complementary form or related primers for the desired DNA sequences, and using hybridization or PCR amplification to assay the archive for the presence of these desired sequence fragments, according to such standard assays are used by those skilled in the art of molecular biology to ascertain the presence of a sequence segment in a complex pool of DNA fragments. The search could report either presence or absence, or could recover the associated fragments containing the search string for complete reading.

The support can be formed according to many suitable methods. For example, the support can be formed by hydroxylating the surface of the low temperature co-fired ceramic support 102. Hydroxylating is typically conducted by exposing the support to an acid or to a base. Examples of suitable acids and bases include hydrochloric acid, sulfuric acid, phosphoric acid, hydrofluoric acid, sodium hydroxide, lithium hydroxyde, potassium hydroxyde, or the like. The hydroxylated surface allows for linker molecule 104 to bond to low temperature co-fired ceramic support 102. Linker molecule 104 can then be reacted with individual nucleotides through phosphoramidite chemical reactions as dictated by the writing devices described herein.

### EXAMPLES

Various aspects of the present invention can be better understood by reference to the following Examples which are offered by way of illustration. The present invention is not limited to the Examples given herein.

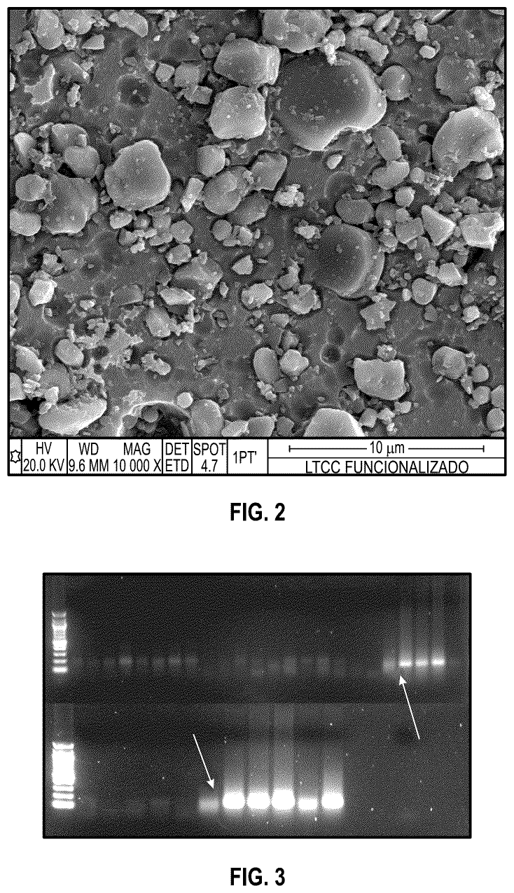

A laboratory-scale protocol is used to demonstrate that DNA can be synthesized using an low temperature co-fired ceramic support. In the protocol an low temperature co-fired ceramic material is functionalized by contacting it with HCl (hydrochloric acid) (0.5 M) and/or NaOH (Sodium hydroxide) (0.5 M) at a temperature between 50° C. to 100° C. for 2 hours to complete the functionalization of the hydroxyl group. Characterization of low temperature co-fired ceramic particles by scanning electron microscopy (SEM). Scanning electron microscopy was performed to verify the surface modification of the low temperature co-fired ceramic. As shown in the image of FIG. 2, there is an increase in the roughness of the surface that received the modification. The linker molecule is functionalized to the low temperature co-fired ceramic support using APTES (3-aminopropyltriethoxysilane) (20 mL) or TMS (Trimethylsilanol) (20 mL), n-propylamine (1 g) and ethanol (90 mL), in a microreactor applying temperature between 50° C. to 100° C. during 2 hours. After this process the low temperature co-fired ceramic support is washed with ethanol and dried under vacuum oven under 80° C. for 24 hours Subsequently, this surface can be subjected to standard oligonucleotide synthesis by the phosphoramidite route.

After the synthesis the oligonucleotide was cleaved out of the surface and the oligonucleotide was amplified by PCR (Polymerase chain reaction). After the amplification the PCR products were subjected to Electrophoresis to evaluate the amplification. As shown in FIG. 3, the amplified oligomer fragment is observed in the gel (FIG. 3—Red arrow) proving that the chemical synthesis of DNA on the modified surface occurred as expected.

The terms and expressions that have been employed are used as terms of description and not of limitation, and there is no intention in the use of such terms and expressions of excluding any equivalents of the features shown and described or portions thereof, but it is recognized that various modifications are possible within the scope of the aspects of the present invention. Thus, it should be understood that although the present invention has been specifically disclosed by specific aspects and optional features, modification and variation of the concepts herein disclosed may be resorted to by those of ordinary skill in the art, and that such modifications and variations are considered to be within the scope of aspects of the present invention.

### Exemplary Aspects

The following exemplary aspects are provided, the numbering of which is not to be construed as designating levels of importance:

Aspect 1 provides a DNA synthesis substrate, the substrate comprising:
a low temperature co-fired ceramic support; and
a linker molecule functionalized to the low temperature co-fired ceramic support.

Aspect 2 provides the substrate of Aspect 1, wherein the low temperature co-fired ceramic support is a monolithic structure.

Aspect 3 provides the substrate of Aspect 1, wherein the low temperature co-fired ceramic support is a layered structure.

Aspect 4 provides the substrate of Aspect 3, wherein the layered structure comprises a first layer of a low temperature co-fired ceramic material and a second layer of a different low temperature co-fired ceramic material.

Aspect 5 provides the substrate of any of Aspects 1-4, wherein the low temperature co-fired ceramic support comprises aluminum, oxygen, silicon, boron, zirconium, lead, magnesium, antimony, neodymium, or a mixture thereof.

Aspect 6 provides the substrate of any of Aspects 1-5, wherein the low temperature co-fired ceramic support comprises:
a mixture of Al₂O₃, forsterite, and borosilicate glass;
a mixture of borosilicate glass, SiO₂, and Al₂O₃;
a mixture of BaO, SiO₂, and Al₂O₃;
CaZrO₃;
a mixture of BaO, B₂O₃, Al₂O₃, CaO, and SiO₂;
a mixture of Nd₂O₃, TiO₂, and SiO₂;
a mixture of PbO, Al₂O₃, and SiO₂;
a mixture of CaO, Al₂O₃, SiO₂, B₂O₃, and Al₂O₃;
a mixture of Al₂O₃, CaO, SiO₂, ZrO₂, MgO, B₂O₃; or a mixture of Al₂O₃, SiO₂, ZrO₂, and MgO.

Aspect 7 provides the substrate of any of Aspects 1-6, wherein a surface of the ceramic support is functionalized.

Aspect 8 provides the substrate of Aspect 7, wherein about 50% to about 100% surface area of the surface is functionalized.

Aspect 9 provides the substrate of any of Aspects 7 or 8, wherein about 75% to about 90% surface area of the surface is functionalized.

Aspect 10 provides the substrate of any of Aspects 7-9, wherein the surface is functionalized with hydroxyl groups.

Aspect 11 provides the substrate of any of Aspects 1-10, wherein the linker molecule comprises a silicon.

Aspect 12 provides the substrate of Aspect 11, wherein the linker molecule comprises a functionalized compound according to Formula I:

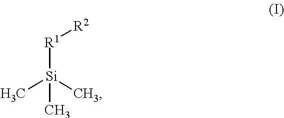

wherein
R¹ is selected from the group consisting of a bond or (C₁-C₂₀)hydrocarbyl;
R² is selected from the group consisting of —OH and N(R³)₂; and at each instance, R³ is independently selected from the group consisting of —H or (C₁-C₂₀)hydrocarbyl.

Aspect 13 provides the substrate of Aspect 12, wherein R¹ are selected from the group consisting of a bond or (C₂-C₇)hydrocarbyl.

Aspect 14 provides the substrate of any of Aspects 12 or 13, wherein R¹ is selected from the group consisting of a bond or (C₂-C₂₀)alkenyl, (C₂-C₂₀)aryl, (C₂-C₂₀)hydroxyl, (C₂-C₂₀)alkyl, or (C₂-C₂₀)cycloalkyl.

Aspect 15 provides the substrate of any of Aspects 1-14, wherein the linker molecule comprises a 3-aminopropyltriethoxysilane, a trimethylsilanol, or both.

Aspect 16 provides the substrate of any of Aspects 1-15, wherein the substrate comprises at least two different linker molecules.

Aspect 17 provides the substrate of any of Aspects 1-16, further comprising an oligonucleotide bonded to the linker molecule.

Aspect 18 provides the substrate of Aspect 17, wherein the oligonucleotide comprises a single stranded DNA.

Aspect 19 provides the substrate of any of Aspects 17 or 18, wherein the oligonucleotide comprises 2 to 300 nucleotides.

Aspect 20 provides a DNA synthesis device comprising:
a reaction chamber; and
the substrate of any of Aspects 1-19, located at least partially within the reaction chamber.

Aspect 21 provides the device of Aspect 20, further comprising:
a second reaction chamber; and
a second substrate of any of Aspects 1-19, located at least partially within the second reaction chamber.

Aspect 26 provides a method of synthesizing a single stranded DNA oligomer, the method comprising:
contacting a first nucleotide with the substrate of any of Aspects 1-19;
forming a bond between the linker and the first nucleotide;
contacting the first nucleotide with a second nucleotide; and forming a bond between the first nucleotide and the second nucleotide.

Aspect 27 provides the method of Aspect 26, further comprising:
contacting the second nucleotide with a third nucleotide; and
forming a bond between the second nucleotide and the third nucleotide.

Aspect 28 provides the method of any of Aspects 26 or 27, further comprising cleaving the single stranded DNA oligomer from the linker.

Aspect 29 provides the method of any of Aspects 26-28, wherein the first nucleotide, second nucleotide, and third nucleotide are phosphoramidites.

Aspect 30 provides a method of making the substrate of any of Aspects 1-19, the method comprising:
hydroxylating a surface of the low temperature co-fired ceramic support;
contacting the hydroxylated surface of the low temperature co-fired ceramic support with the linker molecule;
bonding the linker molecule to a hydroxyl group of the hydroxylated surface of the low temperature co-fired ceramic support.

Aspect 31 provides the method of Aspect 30, further comprising integrating the substrate to an existing platform.

Aspect 32 provides the method of Aspect 31, wherein the existing platform comprises a DNA synthesis machine, a well, a channel or a reaction site.

Aspect 33 provides the method of any of Aspects 30-32, wherein hydroxylating a surface of the low temperature co-fired ceramic support comprises contacting the surface with an acid or a base.

Aspect 34 provides a DNA oligomer formed according to the method of any of Aspects 26-29.

Aspect 35 provides an information storage system, comprising:
a device that synthesizes a single stranded DNA oligomer that encodes a set of information, the device comprising the DNA synthesis substrate of any of Aspects 1-34; and
a reading device that interprets the single stranded DNA oligomer by decoding the interpreted single stranded DNA oligomer into the set of information,
wherein the reading device comprises a molecular electronics sensor that produces distinguishable signals in a measurable electrical parameter of the molecular electronics sensor, when interpreting the single stranded DNA oligomer.

Aspect 36 provides the system of Aspect 35, wherein the set of information is binary.

Aspect 37 provides the system of Aspect 35 or 36, further comprising at least one of error detecting schemes or error correction schemes for minimizing errors within the single stranded DNA oligomer.

Aspect 38 provides the system of Aspect 37, wherein the error detecting schemes are selected from repetition code, parity bits, checksums, cyclic redundancy checks, cryptographic hash functions and hamming codes, and the error correction schemes are selected from automatic repeat request, convolutional codes, block codes, hybrid automatic repeat request and Reed-Solomon codes.

Aspect 39 provides the system of any of Aspects 35-38, wherein the device comprises a CMOS chip based array of actuator pixels for DNA synthesis, the actuator pixels directing voltage/current or light-mediated deprotection within a DNA synthesis reaction comprising phosphoramidite chemistries.

What is claimed is:

1. A DNA synthesis substrate, the substrate comprising:
a low temperature co-fired ceramic support; and
a linker molecule functionalized to the low temperature co-fired ceramic support.

2. The substrate of claim 1, wherein the low temperature co-fired ceramic support is a monolithic structure.

3. The substrate of claim 1, wherein the low temperature co-fired ceramic support is a layered structure.

4. The substrate of claim 3, wherein the layered structure comprises a first layer of a low temperature co-fired ceramic material and a second layer of a different low temperature co-fired ceramic material.

5. The substrate of claim 1, wherein the low temperature co-fired ceramic support comprises aluminum, oxygen, silicon, boron, zirconium, lead, magnesium, antimony, neodymium, or a mixture thereof.

6. The substrate of claim 1, wherein the low temperature co-fired ceramic support comprises:
a mixture of Al₂O₃, forsterite, and borosilicate glass;
a mixture of borosilicate glass, SiO₂, and Al₂O₃;
a mixture of BaO, SiO₂, and Al₂O₃;
CaZrO₃;
a mixture of BaO, B₂O₃, Al₂O₃, CaO, and SiO₂;
a mixture of Nd₂O₃, TiO₂, and SiO₂;
a mixture of PbO, Al₂O₃, and SiO₂;
a mixture of CaO, Al₂O₃, SiO₂, B₂O₃, and Al₂O₃;
a mixture of Al₂O₃, CaO, SiO₂, ZrO₂, MgO, B₂O₃; or
a mixture of Al₂O₃, SiO₂, ZrO₂, and MgO.

7. The substrate of claim 1, wherein a surface of the ceramic support is functionalized.

8. The substrate of claim 7, wherein about 50% to about 100% surface area of the surface is functionalized.

9. The substrate of claim 7, wherein about 75% to about 90% surface area of the surface is functionalized.

10. The substrate of claim 7, wherein the surface is functionalized with hydroxyl groups.

11. The substrate of claim 1, wherein the linker molecule comprises a silicon.

12. The substrate of claim 11, wherein the linker molecule comprises a functionalized compound according to Formula I: 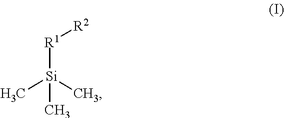
wherein
R¹ is selected from the group consisting of a bond or (C₁-C₂₀)hydrocarbyl;
R² is selected from the group consisting of —OH and N(R³)₂; and
at each instance, R³ is independently selected from the group consisting of —H or (C₁-C₂₀)hydrocarbyl.

13. The substrate of claim 12, wherein R¹ is selected from the group consisting of a bond or (C₂-C₇)hydrocarbyl.

14. The substrate of claim 12, wherein R¹ is selected from the group consisting of a bond or (C₂-C₂₀)alkenyl, (C₂-C₂₀)aryl, (C₂-C₂₀)hydroxyl, (C₂-C₂₀)alkyl, or (C₂-C₂₀)cycloalkyl.

15. The substrate of claim 1, wherein the linker molecule comprises a 3-aminopropyltriethoxysilane, a trimethylsilanol, or both.

16. The substrate of claim 1, wherein the substrate comprises at least two different linker molecules.

17. The substrate of claim 1, further comprising an oligonucleotide bonded to the linker molecule.

18. The substrate of claim 17, wherein the oligonucleotide comprises a single stranded DNA.

19. The substrate of claim 17, wherein the oligonucleotide comprises 2 to 300 nucleotides.

20. A DNA synthesis device comprising:
a reaction chamber; and
the substrate of claim 1, located at least partially within the reaction chamber.

21. The device of claim 20, further comprising:
a second reaction chamber; and
a second substrate of claim 1, located at least partially within the second reaction chamber.

22. The device of claim 21, wherein the reaction chamber and the second reaction chamber are independently a well, a channel, a cartridge, a pore or a reaction site.

23. The device of claim 20, wherein the device is a microdevice or a nanodevice.

24. The device of claim 20, wherein the device is a microarray or a DNA synthesizer.

25. The device of claim 20, wherein the device is an automated device.

26. A method of synthesizing a single stranded DNA oligomer, the method comprising:
contacting a first nucleotide with the substrate of claim 1;
forming a bond between the linker and the first nucleotide;
contacting the first nucleotide with a second nucleotide; and
forming a bond between the first nucleotide and the second nucleotide.

27. The method of claim 26, further comprising:
contacting the second nucleotide with a third nucleotide; and
forming a bond between the second nucleotide and the third nucleotide.

28. The method of claim 26, further comprising cleaving the single stranded DNA oligomer from the linker.

29. The method of claim 26, wherein the first nucleotide, second nucleotide, and third nucleotide are phosphoramidites.

30. A method of making the substrate of claim 1, the method comprising:
hydroxylating a surface of the low temperature co-fired ceramic support;
contacting the hydroxylated surface of the low temperature co-fired ceramic support with the linker molecule;
bonding the linker molecule to a hydroxyl group of the hydroxylated surface of the low temperature co-fired ceramic support.

31. The method of claim 30, further comprising integrating the substrate to an existing platform.

32. The method of claim 31, wherein the existing platform comprises a DNA synthesis machine, a well, a channel or a reaction site.

33. The method of claim 30, wherein hydroxylating a surface of the low temperature co-fired ceramic support comprises contacting the surface with an acid or a base.

34. A DNA oligomer formed according to the method of claim 26.

35. An information storage system, comprising:
a device that synthesizes a single stranded DNA oligomer that encodes a set of information, the device comprising the DNA synthesis substrate of claim 1; and
a reading device that interprets the single stranded DNA oligomer by decoding the interpreted single stranded DNA oligomer into the set of information,
wherein the reading device comprises a molecular electronics sensor that produces distinguishable signals in a measurable electrical parameter of the molecular electronics sensor, when interpreting the single stranded DNA oligomer.

36. The system of claim 35, wherein the set of information is binary.

37. The system of claim 35, further comprising at least one of error detecting schemes or error correction schemes for minimizing errors within the single stranded DNA oligomer.

38. The system of claim 37, wherein the error detecting schemes are selected from repetition code, parity bits, checksums, cyclic redundancy checks, cryptographic hash functions and hamming codes, and the error correction schemes are selected from automatic repeat request, convolutional codes, block codes, hybrid automatic repeat request and Reed-Solomon codes.

39. The system of claim 35, wherein the device comprises a CMOS chip based array of actuator pixels for DNA synthesis, the actuator pixels directing voltage/current or light-mediated deprotection within a DNA synthesis reaction comprising phosphoramidite chemistries.
